(19) United States
(12) Patent Application Publication
(10) Pub. No.: US 2026/0085049 A1
(43) Pub. Date: Mar. 26, 2026

(54) CRYSTAL FORM OF ERGOTHIONEINE

(71) Applicant: NANJING NUTRABUILDING BIO-TECH CO., LTD., Nanjing (CN)

(72) Inventors: Jingwei ZHOU, Nanjing (CN); Yongxiao ZHU, Nanjing (CN); Shawn WELLS, Frisco, TX (US); Xi ZHU, Nanjing (CN); Kylin LIAO, Nanjing (CN)

(21) Appl. No.: 19/409,672

(22) Filed: Dec. 4, 2025

Related U.S. Application Data
(63) Continuation of application No. PCT/CN2024/098613, filed on Jun. 12, 2024.

(30) Foreign Application Priority Data
Jun. 12, 2023 (WO) PCT/CN2023/099657

(51) Int. Cl. C07D 233/84

(52) U.S. Cl. CPC C07D 233/84

(57) ABSTRACT

The invention discloses a crystal form of ergothioneine and a preparation method thereof. The method provided by the invention can efficiently and stably produce anhydrous crystal form of ergothioneine. Moreover, the ergothioneine crystal form of the invention has high purity, high mass fraction, low moisture content and high crystallization yield, so it can have a wide application prospect in the fields of dietary supplements, foods or cosmetics.

### CROSS REFERENCE TO RELATED APPLICATION

This application is a continuation application of International Patent Application No. PCT/CN2024/098613, filed on Jun. 12, 2024, which claims the priority of the International Application No. PCT/CN2023/099657, filed on Jun. 12, 2023, the contents of all of which are incorporated herein by reference in their entirety.

### FIELD OF THE INVENTION

The invention belongs to the technical field of crystallization, in particular to a crystal form of ergothioneine and preparation method thereof.

### BACKGROUND OF THE INVENTION

Ergothioneine (EGT) is a natural rare chiral amino acid which is derived from plants and can be accumulated in animals. It is safe and nontoxic. It is a natural antioxidant, which has many physiological functions, such as detoxification, maintaining DNA growth, anti-radiation, anti-inflammation, anti-aging, protecting nervous system, inhibiting developmental defects, protecting liver and so on. Ergothioneine exists in the whole human body, with the highest content in kidney, liver, red blood cells and semen, which is very important for human health. Clinical research on ergothioneine in cognition, mood, sleep, and the like is under way at home and abroad. In view of its physiological activity, EGT has a wide application prospect in dietary supplements, cosmetics, medicine and other fields.

At present, methods of producing ergothioneine include chemical synthesis, biological extraction and biological fermentation. The ergothioneine prepared by the existing methods is mostly flaky and amorphous, and the crystallinity is not high. Moreover, due to the high solubility of ergothioneine in water, the crystallization yield is generally low. There are many factors that affect the crystallization of ergothioneine, such as supersaturation of solution, purity of ergothioneine in solution, types of inorganic salts and addition amounts thereof, types of anti-solvents and addition amounts thereof, stirring mode and mixing degree, crystallization temperature, etc. Therefore, the preparation of ergothioneine crystals with high yield and purity, short crystallization time, low energy and material consumption and uniform crystal size distribution and the selection of suitable crystal forms are the key contents that those skilled in the art need to study.

### SUMMARY OF THE INVENTION

In the invention, according to the conditions required for the crystal formation of ergothioneine, the anhydrous crystal form of ergothioneine can be stably prepared by controlling the crystallization process, which has the advantages of high yield (≥90%), high purity (>99.5%), short crystallization time, low energy and material consumption, uniform crystal particle size distribution and the like, and the obtained anhydrous crystal has good fluidity and stability, and can be better applied to the fields of dietary supplements, foods, or cosmetics.

In one aspect, the present invention provides an anhydrous crystal form A of ergothioneine, the X-ray powder diffraction pattern of which comprises peaks at diffraction angles (2θ) of 8.6°±0.1°, 19.0°±0.1°, 26.4°±0.1°, and 32.3°±0.1°.

In some embodiments, the X-ray powder diffraction pattern of the anhydrous crystal form A further comprises one or more peaks at diffraction angles (2θ) of 15.6°±0.1°, 20.5°±0.1°, 24.9°±0.1°.

In some embodiments, the X-ray powder diffraction pattern of the anhydrous crystal form A further comprises one or more peaks at diffraction angles (2θ) of 17.3°±0.1°, 22.3°±0.1°, 34.7°±0.1°.

In some embodiments, the X-ray powder diffraction pattern of the anhydrous crystal form A is shown in FIG. 1.

In another aspect, the present invention provides a preparation method of the anhydrous crystal form A as described above, which includes the following steps: dissolving ergothioneine in water to obtain an ergothioneine solution; after concentration, adding one or more monovalent cation salts of formate, acetate, citrate, stirring and balancing the temperature, and adding a water-miscible organic solvent or a mixed solvent of the organic solvent and water to the solution, and continuing the stirring; cooling to obtain an ergothioneine crystal slurry; suction filtering and drying the ergothioneine crystal slurry to obtain the anhydrous crystal form A.

In some embodiments, the ergothioneine used for dissolution can be an amorphous ergothioneine. In some embodiments, the concentrating makes the concentration of the ergothioneine solution exceed 120 g/L. The concentrating can be carried out by rotary evaporation concentration. In some embodiments, after rotary evaporation concentration makes the concentration of the ergothioneine solution exceed 200 g/L, preferably 250 g/L, more preferably 290 g/L, the pH is adjusted to 6-8, preferably 6.5-7.5, more preferably 6.5-7.

In some embodiments, the monovalent cation salt of formate, acetate, citrate is selected from sodium formate, potassium formate, ammonium formate, sodium acetate, potassium acetate, ammonium acetate, sodium citrate, potassium citrate, ammonium citrate; the organic solvent is selected from one or more of lower alkyl alcohol or lower alkyl ketone. In some embodiments, the monovalent cation salt of formate, acetate, citrate is selected from sodium formate, sodium acetate, potassium acetate, ammonium acetate and sodium citrate; the organic solvent is selected from one or more of ethanol, methanol, isopropanol, acetone. In some embodiments, the organic solvent is selected from one or more of ethanol, methanol and isopropanol; the flow rate of the organic solvent is 5-15% BV/h, preferably 10% BV/h.

In some embodiments, the cooling is cooling quickly to 10-30° C., preferably 15-25° C. In some embodiments, the drying can be carried out by vacuum drying.

In another aspect, the present invention provides an anhydrous crystal form B of ergothioneine, the X-ray powder diffraction pattern of which comprises peaks at diffraction angles (2θ) of 15.3°±0.1°, 22.8°±0.1°, 27.7°±0.1°, and 33.5°±0.1°.

In some embodiments, the X-ray powder diffraction pattern of the anhydrous crystal form B further comprises one or more peaks at diffraction angles (2θ) of 11.2°±0.1°, 22.5°±0.1°, 35.4°±0.1°.

In some embodiments, the X-ray powder diffraction pattern of the anhydrous crystal form B further comprises one or more peaks at diffraction angles (2θ) of 12.8°±0.1°, 28.4°±0.1°, 39.9°±0.1°.

In some embodiments, the X-ray powder diffraction pattern of the anhydrous crystal form B is shown in FIG. 2.

In another aspect, the present invention provides a preparation method of the anhydrous crystal form B as described above, which includes the following steps: dissolving ergothioneine in water to obtain an ergothioneine solution; adding a water-miscible organic solvent or a mixed solvent of the organic solvent and water to the solution under stirring, cooling to obtain an ergothioneine crystal slurry; suction filtering and drying the ergothioneine crystal slurry to obtain the anhydrous crystal form B.

In some embodiments, the ergothioneine used for dissolution can be an amorphous ergothioneine. In some embodiments, the dissolution is promoted by any one or a combination of the following methods: heating, adjusting pH to 6-7 or stirring; after dissolution, it is concentrated to make the concentration of the ergothioneine solution exceed 120 g/L. In some embodiments, the heating can be carried out in a water bath at 30-80° C., preferably 35-70° C., and more preferably 40-60° C. In some embodiments, the pH is adjusted to 6-8, preferably 6.5-7.5, more preferably 6.5-7. In some embodiments, pH adjustment can be carried out in a conventional manner, such as using sodium hydroxide or hydrochloric acid solution. In some embodiments, dissolution can be promoted by stirring at 50-300 rpm, preferably 100-200 rpm, and more preferably 150 rpm. In some embodiments, the concentrating is carried out under vacuum at 40-80° C., preferably 50-70° C., more preferably 60-70° C., to make the concentration of ergothioneine solution exceed 200 g/L, preferably 250 g/L, more preferably 290 g/L.

In some embodiments, the organic solvent is selected from one or more of lower alkyl alcohol or lower alkyl ketone. In some embodiments, the organic solvent is selected from one or more of ethanol, methanol, isopropanol, acetone, and is preferably selected from one or more of ethanol, methanol, isopropanol. In some embodiments, the water-miscible organic solvent or the mixed solvent of the water-miscible organic solvent and water can be added by feeding, such as by a peristaltic pump. In some embodiments, the addition rate of the water-miscible organic solvent or the mixed solvent of the water-miscible organic solvent and water is below 60%/h, preferably below 55%/h, more preferably below 50%/h of the volume of the ergothioneine solution; the volume of the water-miscible organic solvent or the mixed solvent of the water-miscible organic solvent and water is 0.1-5 times, preferably 0.3-2 times, the volume of the ergothioneine solution.

In some embodiments, after adding the water-miscible organic solvent or the mixed solvent of the water-miscible organic solvent and water, cooling is carried out to not higher than 20° C. In some embodiments, after adding the water-miscible organic solvent or the mixed solvent of the water-miscible organic solvent and water, cooling is carried out to not higher than 20° C. and it is maintained for more than 0.5 hour, or to not higher than 15° C., preferably 12° C. and it is maintained for more than 0.5 hour, preferably 1 hour, more preferably 1.5 hour. In some embodiments, the drying can be carried out by vacuum drying at 50-70° C., preferably 55-65° C.

In another aspect, the present invention provides an anhydrous crystal form C of ergothioneine, the X-ray powder diffraction pattern of which comprises peaks at diffraction angles (2θ) of 11.2°±0.1°, 18.1°±0.1°, 24.0°±0.1°, and 26.9°±0.1°.

In some embodiments, the X-ray powder diffraction pattern of the anhydrous crystal form C further comprises one or more peaks at diffraction angles (2θ) of 12.8°±0.1°, 19.8°±0.1°, 24.6°±0.1°.

In some embodiments, the X-ray powder diffraction pattern of the anhydrous crystal form C further comprises one or more peaks at diffraction angles (2θ) of 15.6°±0.1°, 19.0°±0.1°, 21.6°±0.1°.

In some embodiments, the X-ray powder diffraction pattern of the anhydrous crystal form C is shown in FIG. 3.

In another aspect, the present invention provides a preparation method of the anhydrous crystal form C as described above, which includes the following steps: dissolving ergothioneine in water, heating to 60-100° C., dropwise adding a water-miscible organic solvent or a mixed solvent of the water-miscible organic solvent and water, and stirring; then rapidly cooling to 10-30° C., and continuously stirring, to obtain the anhydrous crystal form C.

In some embodiments, the organic solvent is selected from one or more of lower alkyl alcohol or lower alkyl ketone, and is preferably selected from one or more of ethanol, methanol, isopropanol, acetone. In some embodiments, the heating is heating to 70-95° C., preferably 75-90° C.; the organic solvent is selected from one or more of ethanol, methanol and isopropanol; the cooling is cooling to 15-25° C. quickly.

In some embodiments, the anhydrous crystal form as described above can be used for preparing food, beverage, supplement, nutritional product and cosmetic.

In another aspect, the present invention provides a composition comprising an effective amount of the anhydrous crystal form as described above, and a pharmaceutically acceptable carrier.

In some embodiments, the composition can be used for preparing food, beverage, supplement, nutritional product and cosmetic.

Compared with the prior art, the method provided by the invention can efficiently and stably produce anhydrous crystal form of ergothioneine. Moreover, the ergothioneine crystal form of the invention has high purity, which can reach the mass fraction of more than 99.5%, low moisture content (<0.3%) and high crystallization yield (≥90%), so it can have a wide application prospect in the fields of dietary supplements, foods or cosmetics.

### BRIEF DESCRIPTION OF THE DRAWINGS

FIG. 1 shows an XRPD pattern of the anhydrous crystal form A of the present invention.

FIG. 2 shows an XRPD pattern of the anhydrous crystal form B of the present invention.

FIG. 3 shows an XRPD pattern of the anhydrous crystal form C of the present invention.

FIG. 4 shows a DSC diagram of the anhydrous crystal form A of the present invention.

FIG. 5 shows a DSC diagram of the anhydrous crystal form C of the present invention.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the terms “about” and “approximately” provide numerical flexibility by providing endpoints where a given value can be “slightly above” or “less than”. The flexibility of this term can be determined by specific variables and based on experience and related descriptions herein within the knowledge of those skilled in the art.

As used herein, the term “administration” refers to the process of delivering a disclosed crystal form or active ingredient to a subject. The crystal forms of the invention can be administered in a variety of suitable ways, including oral administration, intragastrical administration, parenteral administration (e.g., intravenous and intraarterial as well as other suitable parenteral routes), topical administration and the like, so as to exert the desired effects. The crystal form of the present invention can be administered to a subject at an effective dose and/or at an effective frequency. In some embodiments, multiple doses of the crystal form are administered over a period of time. The administration frequency, duration, etc. of the crystal form can vary depending on any of a variety of factors, including subject response, desired effect, etc.

As used herein, the term “effective amount” refers to the amount required to achieve an effect as taught herein. The amount to be administered can vary according to factors such as the degree of susceptibility of the individual, the age, sex, and weight of the individual, idiosyncratic responses of the individual, and the like. In accordance with the present disclosure, a suitable single dose size is a dose that is capable of achieve the above effects when administered one or more times over a suitable time period.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

In some embodiments, the crystal form of the present invention can be prepared into a composition together with an alimentarily or pharmaceutically acceptable carrier. In the present invention, the application form of providing composition involves liquid or solid fillers, diluents, excipients, solvents or encapsulate materials. Each carrier must be “acceptable”, which means that it is compatible with other components of the composition and harmless to the subject, that is, suitable for consumption or nutritionally acceptable.

In some embodiments, the crystal forms of the present invention can be administered together with other supplements, such as vitamins, minerals, nootropics, and others known in the art.

The following examples are illustrative of select embodiments of the present invention and are not meant to limit the scope of the invention.

The experimental methods described in the following examples are all conventional methods unless otherwise specified. The reagents and materials can be obtained from commercial sources unless otherwise specified. The rotational speed of stirring can be an appropriate rotational speed, such as 150 rpm. Ethanol is anhydrous ethanol solution. The ergothioneine content of amorphous ergothioneine can be 98%.

### Example 1. Preparation of Anhydrous Crystal Form A

300 g of amorphous ergothioneine was dissolved in 1.5 L of pure water, and the volume of the solution was concentrated to 1 L by rotary evaporation concentration, so that the concentration of ergothioneine was 300 g/L. Then, 1.8 g of sodium acetate was added to the solution, stirred and dissolved, and its pH was adjusted to 7.0. Then, anhydrous ethanol was added to the solution through a peristaltic pump at a flow rate of 10% BV/h, and a total of 0.5 BV (about 0.5 L) of anhydrous ethanol was added. After the addition of ethanol, stirring was continued for 1 hour, then the solution was cooled by 20° C. at a cooling rate of 5° C./h, and then the crystal was filtered and dried to obtain 276 g of anhydrous ergothioneine crystal form with a yield of 92% and a content of 99.9%.

### Example 2. Preparation of Anhydrous Crystal Form B

150 g of amorphous ergothioneine was dissolved in 1 L of pure water, the dissolution was accelerated by stirring and water bath (45-50° C.), then the pH of the solution was adjusted to 7.0, and then the concentration of ergothioneine was increased to 300 g/L by vacuum concentration (65° C.). The concentrated solution was transferred to a 2 L crystallization tank, and anhydrous ethanol was added with constant stirring to the crystallization tank by a peristaltic pump at a flow rate of 100 mL/h, with a total addition of 1000 mL. After the completion of anhydrous ethanol addition, the temperature was cooled to 5° C. through the jacket of the crystallization tank, and the temperature was maintained for 1 hour. Then the stirring was stopped, and the ergothioneine crystal slurry in the crystallization tank was discharged. The crystal was suction filtered, and dried in a vacuum drying oven at 60° C., to obtain 144 g of anhydrous ergothioneine crystal form with a yield of 96% and a content of 99.8%.

### Example 3. Preparation of Anhydrous Crystal Form B

200 g of amorphous ergothioneine was dissolved in 1 L of pure water, the dissolution was accelerated by stirring and water bath (50-60° C.), then the pH of the solution was adjusted to 7.0, and then the concentration of ergothioneine was increased to 350 g/L by vacuum concentration (65° C.). The concentrated solution was transferred to a 2 L crystallization tank, and methanol was added with constant stirring to the crystallization tank by a peristaltic pump at a flow rate of 200 mL/h, with a total addition of 1000 mL. After the completion of methanol addition, the temperature was cooled to 10° C. through the jacket of the crystallization tank, and the temperature was maintained for 1 hour. Then the stirring was stopped, and the ergothioneine crystal slurry in the crystallization tank was discharged. The crystal was suction filtered, and dried in a vacuum drying oven at 60° C., to obtain 190 g of anhydrous ergothioneine crystal form with a yield of 95% and a content of 99.8%.

### Example 4. Preparation of Anhydrous Crystal Form B

50 g of amorphous ergothioneine was dissolved in 0.5 L of pure water, the dissolution was accelerated by stirring and water bath (40-50° C.), then the pH of the solution was adjusted to 7.0, and then the concentration of ergothioneine was increased to 310 g/L by vacuum concentration (65° C.). The concentrated solution was transferred to a 2 L crystallization tank, and isopropanol was added with constant stirring to the crystallization tank by a peristaltic pump at a flow rate of 80 mL/h, with a total addition of 320 mL. After the completion of isopropanol addition, the temperature was cooled to 12° C. through the jacket of the crystallization tank, and the temperature was maintained for 1 hour. Then the stirring was stopped, and the ergothioneine crystal slurry in the crystallization tank was discharged. The crystal was suction filtered, and dried in a vacuum drying oven at 60° C., to obtain 45.5 g of anhydrous ergothioneine crystal form with a yield of 91% and a content of 99.7%.

### Example 5. Preparation of Anhydrous Crystal Form C

1 g of ergothioneine was dissolved in 1 mL of water, heated to 80-90° C. for dissolution, cooled to 35° C., and 5 mL of methanol was added dropwise; stirred for 30 min. Then it was rapidly cooled to 20° C., and continuously stirred for 30 min. Finally, 0.95 g anhydrous ergothioneine crystal form was obtained with a yield of 95% and a content of 99.9%.

### Comparative Example 1

75 g of ergothioneine was dissolved in 0.5 L of pure water by stirring and water bath (30-35° C.), and the solution was transferred to a 2 L crystallization tank, then stirred at 25° C. for 1 hour. Then anhydrous ethanol was added to the crystallization tank by a peristaltic pump at a flow rate of 100 mL/h, with a total addition of 1000 mL. After the completion of anhydrous ethanol addition, the temperature was cooled to 10° C. through the jacket of the crystallization tank, and the temperature was maintained for 1 hour. Then the stirring was stopped, and the ergothioneine crystal slurry in the crystallization tank was discharged. The crystal was suction filtered, and dried in a vacuum drying oven at 60° C., to obtain 52.5 g of ergothioneine solid as an amorphous form with a yield of 70% and a content of 98.5%.

X-ray diffraction (XRD), differential scanning calorimetry (DSC), elemental analysis, particle size analysis, quantitative nuclear magnetic resonance (NMR), Raman spectroscopy, infrared spectroscopy (IR), TGA, IPC-MS, DVS and other tests were carried out on the crystal forms prepared in the Examples.

### X-Ray Diffraction

X-ray powder diffraction patterns were obtained by using SmartLab 3 KW X-ray powder diffractometer under the following conditions: diffraction line: Cu_K-beta (40 KV, 40 mA), scanning rate: 20.00 deg/min, and scanning range: 5°˜40°.

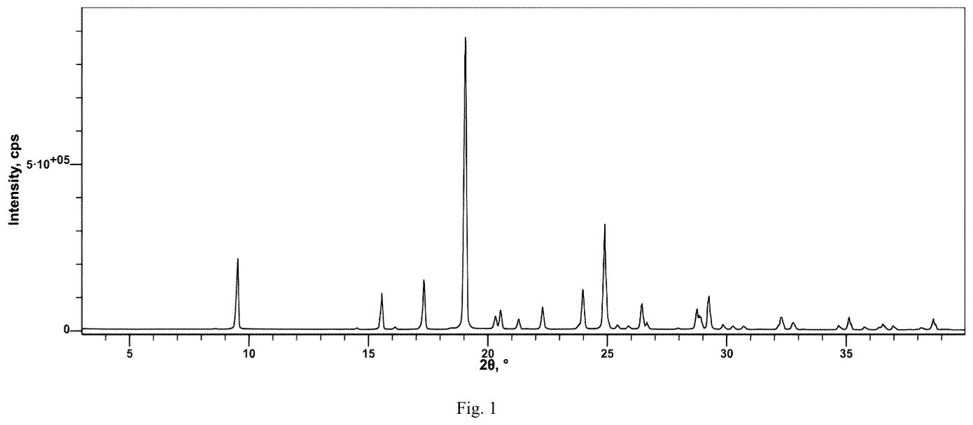

The XRPD pattern of the anhydrous crystal form A obtained in Example 1 is shown in FIG. 1, and the XRPD data is shown in Table 1.

TABLE 1

| | | relative intensity |
|---|---|---|
| position (2θ, °) | spacing (d, Å) | (%) |
| 8.6 | 10.3 | 0.3 |
| 9.5 | 9.3 | 31.7 |
| 14.5 | 6.1 | 0.5 |
| 15.6 | 5.7 | 15.0 |
| 16.1 | 5.5 | 1.0 |
| 17.3 | 5.1 | 24.1 |
| 19.0 | 4.7 | 100.0 |
| 19.1 | 4.6 | 59.1 |
| 20.3 | 4.4 | 6.9 |
| 20.5 | 4.3 | 10.1 |
| 21.3 | 4.2 | 5.6 |
| 22.3 | 4.0 | 11.8 |
| 23.8 | 3.7 | 1.8 |
| 24.0 | 3.7 | 21.2 |
| 24.9 | 3.6 | 57.7 |
| 25.4 | 3.5 | 2.0 |
| 25.9 | 3.4 | 2.1 |
| 26.4 | 3.4 | 14.2 |
| 26.7 | 3.3 | 3.2 |
| 28.0 | 3.2 | 1.0 |
| 28.8 | 3.1 | 10.6 |
| 28.9 | 3.1 | 7.1 |
| 29.2 | 3.1 | 18.4 |
| 29.8 | 3.0 | 2.7 |
| 30.3 | 3.0 | 1.9 |
| 30.7 | 2.9 | 2.0 |
| 32.3 | 2.8 | 9.0 |
| 32.8 | 2.7 | 4.8 |
| 34.7 | 2.6 | 1.9 |
| 35.1 | 2.6 | 6.5 |

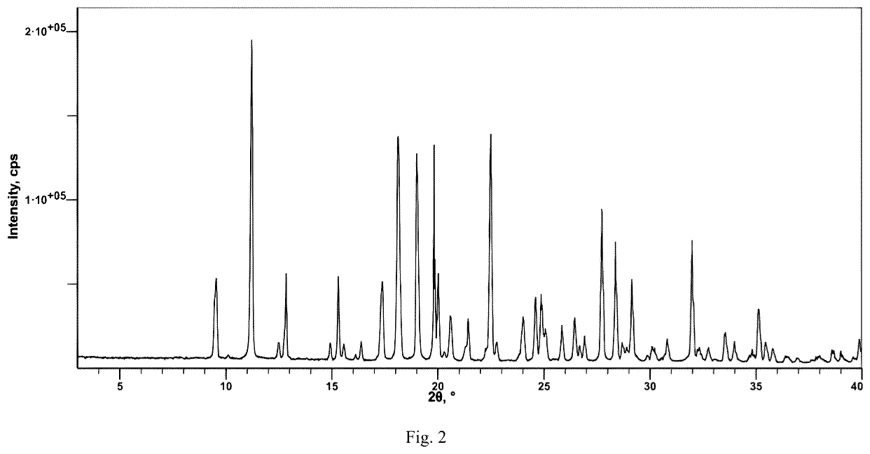

The XRPD pattern of the crystal form B obtained in Example 2 is shown in FIG. 2, and the XRPD data is shown in Table 2.

TABLE 2

| | | relative intensity |
|---|---|---|
| position (2θ, °) | spacing (d, Å) | (%) |
| 9.5 | 9.3 | 31.6 |
| 11.2 | 7.9 | 100.0 |
| 12.8 | 6.9 | 23.0 |
| 15.3 | 5.8 | 20.0 |
| 17.4 | 5.1 | 31.9 |
| 18.1 | 4.9 | 96.2 |
| 19.0 | 4.7 | 71.1 |
| 19.8 | 4.5 | 33.9 |
| 20.0 | 4.4 | 28.2 |
| 20.6 | 4.3 | 17.5 |
| 21.4 | 4.1 | 15.6 |
| 22.5 | 4.0 | 77.2 |
| 22.8 | 3.9 | 6.6 |
| 24.0 | 3.7 | 14.4 |
| 24.6 | 3.6 | 20.5 |
| 24.9 | 3.6 | 22.2 |
| 25.1 | 3.6 | 12.8 |
| 25.8 | 3.4 | 12.2 |
| 26.4 | 3.4 | 15.7 |
| 26.9 | 3.3 | 7.6 |
| 27.7 | 3.2 | 48.1 |
| 28.4 | 3.1 | 36.2 |
| 29.1 | 3.1 | 26.1 |
| 30.8 | 2.9 | 9.8 |
| 32.0 | 2.8 | 37.9 |
| 32.3 | 2.8 | 8.1 |
| 33.5 | 2.7 | 11.7 |
| 35.1 | 2.6 | 20.8 |
| 35.4 | 2.5 | 7.3 |
| 39.9 | 2.3 | 8.1 |

The XRPD results of the crystal forms prepared in Examples 3 and 4 are substantially the same as those in Example 2.

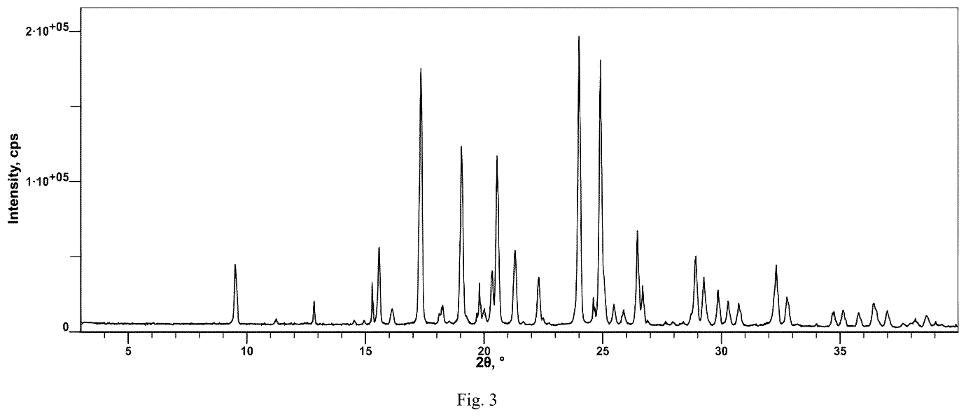

The XRPD pattern of the crystal form C obtained in Example 5 is shown in FIG. 3, and the XRPD data is shown in Table 3.

TABLE 3

| | | relative intensity |
|---|---|---|
| position (2θ, °) | spacing (d, Å) | (%) |
| 9.5 | 9.3 | 18.8 |
| 11.2 | 7.9 | 1.2 |
| 12.8 | 6.9 | 4.2 |
| 14.5 | 6.1 | 0.8 |
| 14.9 | 5.9 | 0.6 |
| 15.3 | 5.8 | 5.4 |
| 15.6 | 5.7 | 23.5 |
| 16.1 | 5.5 | 5.3 |
| 17.2 | 5.1 | 23.6 |
| 17.3 | 5.1 | 66.5 |
| 18.1 | 4.9 | 1.8 |
| 18.2 | 4.9 | 7.0 |
| 19.0 | 4.7 | 64.6 |
| 19.7 | 4.5 | 1.5 |
| 19.8 | 4.5 | 7.2 |
| 20.0 | 4.4 | 5.0 |
| 20.3 | 4.4 | 14.8 |
| 20.5 | 4.3 | 60.0 |
| 21.3 | 4.2 | 28.1 |
| 21.6 | 4.1 | 0.8 |
| 22.3 | 4.0 | 16.3 |
| 22.5 | 4.0 | 1.6 |
| 24.0 | 3.7 | 100.0 |
| 24.6 | 3.6 | 5.2 |
| 24.9 | 3.6 | 99.5 |
| 25.5 | 3.5 | 5.5 |
| 25.9 | 3.4 | 4.9 |
| 26.5 | 3.4 | 30.3 |
| 26.7 | 3.3 | 11.0 |
| 26.9 | 3.3 | 0.9 |

### Differential Scanning Calorimetry

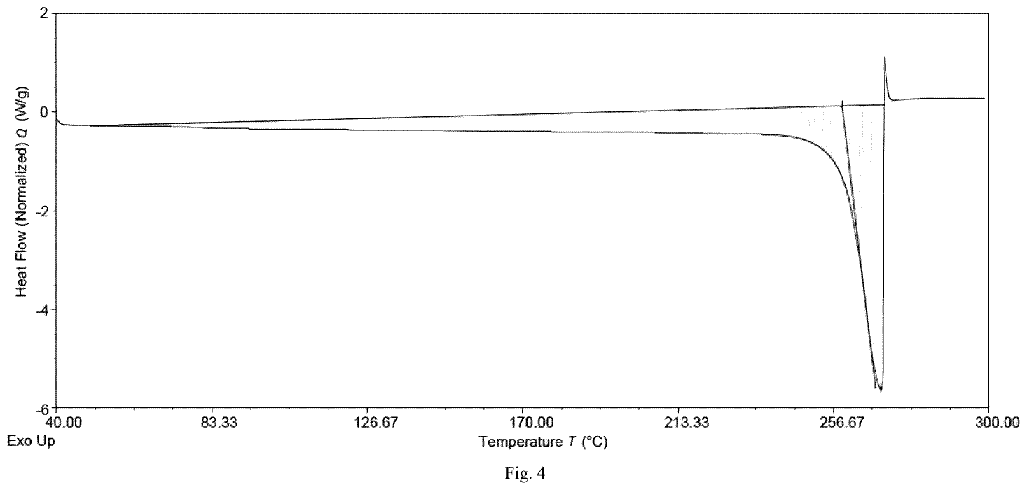

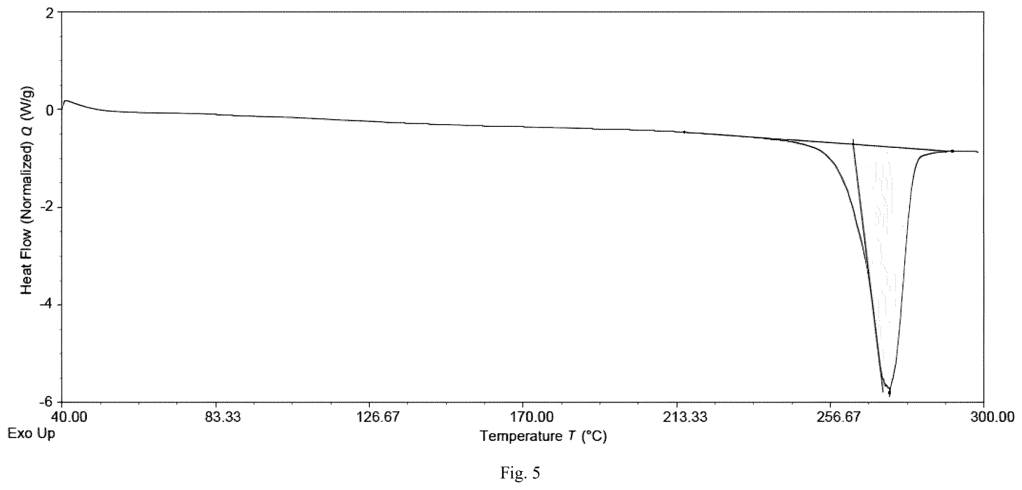

Differential scanning calorimetry (DSC) was carried out with TA Q2000 module with thermal analysis controller, and data were collected and analyzed with TA Instruments Thermal Solutions software. About 1-5 mg of the sample was accurately weighed into a special aluminum crucible with a lid, and the sample was analyzed from 40° C. to about 300° C. at 10° C./min using a linear heating device. During use, the DSC chamber was purged with dry nitrogen. As shown in FIG. 4, the differential scanning calorimetry (DSC) curve of the anhydrous crystal form A of Example 1 contains an endothermic peak of 269.61° C., with an error margin of 3° C. As shown in FIG. 5, the differential scanning calorimetry (DSC) curve of the crystal form C of Example 5 contains an endothermic peak of 273.30° C., with an error margin of 3° C.

### Elemental Analysis

The crystal form C of Example 5 was subjected to elemental analysis by an elemental analyzer: C, 47.01%; H, 6.23%; N, 18.30%; S, 13.59%. The elemental analysis results of anhydrous crystal form A prepared in Example 1 and crystal form B prepared in Examples 2-4 are substantially the same as those in Example 5.

### Quantitative NMR

The ¹H NMR spectra of anhydrous crystal form A of Example 1 were recorded in a spectrometer: ¹H NMR δ (ppm) 6.71, 6.67, 6.25, 4.70, 3.83, 3.79, 3.12.

The ¹H NMR spectra of crystal form C of Example 5 were recorded in a spectrometer: ¹H NMR (400 MHz, deuterium oxide) δ (ppm) 6.71, 6.67, 6.25, 4.70, 3.83, 3.79, 3.14.

The NMR results of crystal form B prepared in Examples 2-4 are substantially the same as those in Examples 1 and 5.

Other crystal forms of the present invention were characterized in a similar manner.

### Analysis and Comparison of Properties

Using crude ergothioneine as raw material, three crystal forms A, B and C were prepared respectively, and their properties were compared with those of amorphous ergothioneine. The results are shown in Table 4:

TABLE 4

| | Color | | | HPLC | | | |
|---|---|---|---|---|---|---|---|
| | and | | Content | purity | | | Moisture |
| | appearance | Odor | (%) | (%) | Absorbance | Hygroscopicity | (%) |
| Crystal | White | None | 99.7 | 99.9 | 0.005 | Stable | 0.07 |
| form A | transparent | | | | | | |
| | crystal | | | | | | |
| Crystal | White | None | 99.6 | 99.9 | 0.006 | Stable | 0.10 |
| form B | transparent | | | | | | |
| | crystal | | | | | | |
| Crystal | White | None | 99.8 | 99.9 | 0.002 | Stable | 0.09 |
| form C | transparent | | | | | | |
| | crystal | | | | | | |
| Amorphous | Yellowish, | Obvious | 98.2 | 99.1 | 0.105 | Easily | 1.29 |
| | flaky | fishy | | | | hygroscopic | |
| | solid | smell | | | | | |

* 10% aqueous solution was prepared and determined at 400 nm.

The preparation methods of ergothioneine usually include chemical synthesis, enzyme catalysis and fermentation, which is the mainstream production method at present because of its low cost and easy industrial scale-up. However, the color of the fermentation broth is dark and the impurity content is high. After purification and concentration, the solution still has a certain color, and the crude ergothioneine after the first crystallization is usually yellow.

As can be seen from the data in Table 4, the three crystal forms prepared in the examples of the present invention are all white transparent crystals, and the absorbance of their aqueous solution at 400 nm is less than 0.01, and they are odorless. The amorphous ergothioneine reference substance prepared from the same raw material is yellowish in color, and its absorbance is 0.105, which is much higher than several crystal forms provided by the invention. In addition, the amorphous ergothioneine has obvious fishy smell because of its low content and purity. In addition, the amorphous ergothioneine has strong hygroscopicity, and the moisture reaches 1.29%, so it is not easy to store for a long time. Whereas, several crystal forms prepared by the examples of the invention are all anhydrous crystals, and all have a moisture that is less than 0.2%, which is not easily hygroscopic and is beneficial to the stability of long-term storage.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. An anhydrous crystal form A of ergothioneine, wherein the X-ray powder diffraction pattern of the anhydrous crystal form A comprises peaks at diffraction angles (2θ) of 8.6°±0.1°, 19.0°±0.1°, 26.4°±0.1°, and 32.3°±0.1°.

2. The anhydrous crystal form A of claim 1, wherein the X-ray powder diffraction pattern of the anhydrous crystal form A further comprises one or more peaks at diffraction angles (2θ) of 15.6°±0.1°, 20.5°±0.1°, 24.9°±0.1°.

3. The anhydrous crystal form A of claim 1, wherein the X-ray powder diffraction pattern of the anhydrous crystal form A further comprises one or more peaks at diffraction angles (2θ) of 17.3°±0.1°, 22.3°±0.1°, 34.7°±0.1°.

4. The anhydrous crystal form A of claim 1, wherein the X-ray powder diffraction pattern of the anhydrous crystal form A is shown in FIG. 1.

5. An anhydrous crystal form B of ergothioneine, wherein the X-ray powder diffraction pattern of the anhydrous crystal form B comprises peaks at diffraction angles (2θ) of 15.3°±0.1°, 22.8°±0.1°, 27.7°±0.1°, and 33.5°±0.1°.

6. The anhydrous crystal form B of claim 5, wherein the X-ray powder diffraction pattern of the anhydrous crystal form B further comprises one or more peaks at diffraction angles (2θ) of 11.2°±0.1°, 22.5°±0.1°, 35.4°±0.1°.

7. The anhydrous crystal form B of claim 5, wherein the X-ray powder diffraction pattern of the anhydrous crystal form B further comprises one or more peaks at diffraction angles (2θ) of 12.8°±0.1°, 28.4°±0.1°, 39.9°±0.1°.

8. The anhydrous crystal form B of claim 5, wherein the X-ray powder diffraction pattern of the anhydrous crystal form B is shown in FIG. 2.

9. An anhydrous crystal form C of ergothioneine, wherein the X-ray powder diffraction pattern of the anhydrous crystal form C comprises peaks at diffraction angles (2θ) of 11.2°±0.1°, 18.1°±0.1°, 24.0°±0.1°, and 26.9°±0.1°.

10. The anhydrous crystal form C of claim 9, wherein the X-ray powder diffraction pattern of the anhydrous crystal form C further comprises one or more peaks at diffraction angles (2θ) of 12.8°±0.1°, 19.8°±0.1°, 24.6°±0.1°.

11. The anhydrous crystal form C of claim 9, wherein the X-ray powder diffraction pattern of the anhydrous crystal form C further comprises one or more peaks at diffraction angles (2θ) of 15.6°±0.1°, 19.0°±0.1°, 21.6°±0.1°.

12. The anhydrous crystal form C of claim 9, wherein the X-ray powder diffraction pattern of the anhydrous crystal form C is shown in FIG. 3.
